(19) United States
(12) Patent Application Publication
(10) Pub. No.: US 2003/0013863 A1
(43) Pub. Date: Jan. 16, 2003

(54) High resolution DNA size standards

(76) Inventors: Wallace G. Buchholz, Anchorage, AK (US); Stephen J. Miller, Anchorage, AK (US)

Correspondence Address: Carla Mattix, Division of General Law, Office of the Solicitor, U.S. Department of the Interior, 1849 C Street NW, MS 6531, Washington, DC, 20240, US

(21) Appl. No.: 09/909,806

(22) Filed: Jul. 16, 2001

(51) Int. Cl. C12P 19/34; C07H 21/04

(52) U.S. Cl. 536/23.1; 435/91.1

(57) ABSTRACT

A method for producing DNA size standards includes using a DNA template having multiple copies of a nucleotide motif sequence. A dideoxy sequencing reaction is prepared which uses the DNA template. In the sequencing reaction, one dideoxy nucleotide terminator is included. The synthesized DNA size standard comprises a mixture of DNA fragments. Each DNA fragment is formed of a respective number of copies of the nucleotide motif. In one further embodiment, each DNA fragment varies in length, from a next shorter or a next longer DNA fragment, by the length of one nucleotide motif sequence.

### BACKGROUND OF THE INVENTION

1. Field of the Invention

The present invention relates to DNA size standards, and in particular, to a method of preparing a DNA size standard comprising a mixture of DNA fragments formed from a varying number of copies of a nucleotide motif sequence.

2. Background of the Invention

DNA size standards are used in genetic studies and forensic applications to determine the relative sizes of nucleic acid fragments in a sample. Typically, commercially available DNA standards include DNA fragments of varying lengths (i.e., number of nucleotide bases). For example, a DNA standard may include fragments that differ by ten nucleotides from 70 to 100 nucleotides (e.g., 70, 80, etc.) and then differ by 20 nucleotides up to 400 nucleotides (e.g., 120, 140, etc).

The DNA size standards typically migrate at a rate corresponding to their size when electrophoresed through a gel matrix. However, secondary structure (i.e. the structure resulting from folding and/or bonding of the nucleotides with each other in a DNA fragment) of the respective DNA fragments will affect the rate at which the fragments migrate. If the DNA size standards fragments include secondary structure, these fragments will migrate aberrantly through an electrophoretic gel.

One disadvantage of currently available commercial DNA size standards is that the DNA size standards do not provide a narrow enough size difference between the DNA fragments to be fully useful in population genetic studies. In population genetic studies, the sizes (i.e., lengths) of different alleles are compared with one another. The size between alleles may differ by as few as one or two nucleotides. In order to properly compare alleles, an accurate relative allele size must be determined. Therefore, it is advantageous to use a DNA size standard with a narrow size difference between fragments. However, as indicated above, typical DNA size standards comprise DNA fragments differing by between 10 to 20 nucleotides in length. Consequently, these DNA size fragments fail to provide the accuracy necessary to size and compare nucleic acid samples (e.g., alleles) which may vary from each other by as few as one or two nucleotides.

A second disadvantage with commercially available size DNA standards is that several fragments typically migrate aberrantly. The aberrant migration of various fragments hampers accurate sizing of nucleic acid samples. One possible explanation for the aberrant migration of the DNA fragments is that the aberration may be due to secondary structure of the respective DNA fragments.

### BRIEF SUMMARY OF THE INVENTION

In accordance with the present invention, a method is provided for preparing DNA sizing standards which produces a mixture of DNA fragments of varying lengths and being formed of a varying number of copies of a nucleotide motif sequence. The motif sequence may be of any sequence and length, but typically the motif sequence is two to six nucleotides in length and includes one unique nucleotide. For example, the motif sequences may include CA, GTA, AACT, or AATA.

The fragments are synthesized from a dideoxy sequencing reaction using a DNA template which comprises multiple copies of the nucleotide motif sequence. The dideoxy sequencing reaction includes one dideoxy nucleotide terminator. The result of the dideoxy sequencing reaction is a DNA size standard formed of a mixture of DNA fragments of a varying number of copies of the nucleotide motif sequence.

The mixture of DNA fragments provide a nucleotide ladder with the size difference between bands being equal to that of the motif sequence length. For example, if the nucleotide motif sequence is two nucleotides long, a two nucleotide ladder will be formed. However, if the nucleotide motif sequence is three nucleotides in length, a three nucleotide ladder will be synthesized.

According to one aspect of the present invention, a method of producing a DNA size standard comprises providing a DNA template having multiple copies of a nucleotide motif sequence. A dideoxy sequencing reaction is prepared using the DNA template and one dideoxy nucleotide terminator. In one embodiment, the dideoxy nucleotide is selected from the group consisting of dideoxy ATP, dideoxy GTP, dideoxy CTP and dideoxy TTP.

According to another aspect of the invention, a DNA size standard comprises a mixture of DNA fragments in which each DNA fragment is formed of a primer, 5′ sequence and a respective number of copies of a nucleotide motif. In one embodiment, the DNA fragments vary in length from a next shorter DNA fragment or a next longer DNA fragment by one copy of the motif.

An advantage of the present invention is a DNA size standard comprising fragments which differ in length equal to the length of the motif sequence. For example, if the motif sequence is two nucleotides long, the DNA fragments will differ by two nucleotides.

An additional advantage of the present invention is a DNA size standard which provides for a more accurate measurement of DNA samples. The size of the motif sequence can be selected to produce DNA fragments that differ in length by as little as one or two nucleotide bases. A DNA standard with fragments that differ by a small number of bases, (e.g., one or two nucleotides), results is a higher resolution (i.e. more precise) measuring of the size of a DNA sample, as compared with a DNA standard having a larger difference between DNA fragments. The enhanced resolution is due to more DNA fragments within a given DNA size range from which to measure a DNA sample.

For example, if the DNA size range is between 70 to 80 nucleotide bases and the motif sequence chosen is two nucleotides long, the DNA size standard will include DNA fragments within the 70 to 80 nucleotide range of 70, 72, 74, 76, 78, and 80 nucleotide bases. A DNA size standard having fragments every two bases provides higher resolution and more accurate DNA sample measurement than a DNA size standard having fragments with larger difference, e.g., 10 to 20 bases.

An additional advantage of the present invention is DNA size standard fragments which lack secondary structure. Motifs may be selected which will not result in secondary structure. As a result, when these DNA fragments are run in an electrophoretic gel, the DNA fragments will migrate at a rate inversely proportional to their respective sizes. Consequently, the DNA size standard of the present invention provides accurate and consistent sizing of DNA samples that can be used to compare sizes of various DNA samples across gels and across platforms.

An object of the present invention is to use a DNA template formed of multiple copies of a nucleotide motif to synthesize a DNA size standard.

An additional object of the present invention is to use microsatellite loci as DNA templates to synthesize DNA size standards.

Another object of the present invention is to provide a DNA size standard formed of DNA fragments which lack secondary structure.

Yet another object of the present invention is to provide a DNA size standard of fragments that differ in length by one copy of the motif sequence.

### BRIEF DESCRIPTION OF THE DRAWING

FIG. 1 illustratively depicts a dideoxy sequencing reaction according to the present invention;

FIG. 2 illustratively depicts a DNA size standard formed of DNA fragments run on an electrophoresis gel according to the present invention; and

FIG. 3 illustratively depicts another DNA size standard formed of DNA fragments run on an electrophoresis gel according to the present invention.

### DETAILED DESCRIPTION OF THE INVENTION

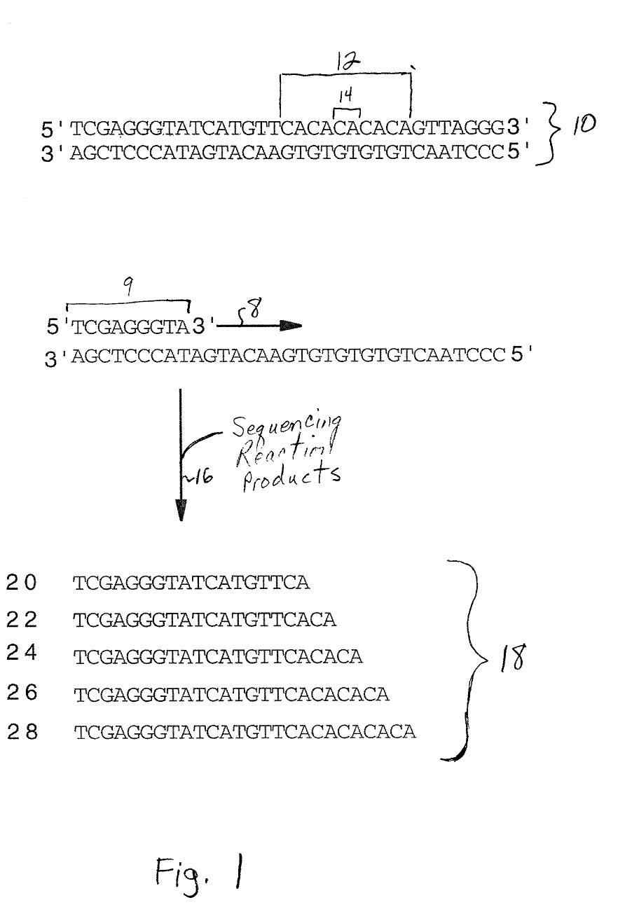

Referring now to FIG. 1, a double standard DNA sequence, denoted 10, is show which comprises a microsatellite locus 12. DNA sequence 10 is a basic sequence used for illustrative purposes and is used to simplify the explanation of the present invention.

Microsatellite locus 12 is composed of five copies of CA nucleotide motif sequence 14. Unique DNA sequences (i.e. non-motif, repeating sequences) flank both the 5′ and 3′ ends of the microsatellite locus 12. In the example illustrated, DNA sequence TCGAGGGTATCATGTT flanks the 5′ end and DNA sequence GTTAGGG flanks the 3′ end of microsatellite locus 12.

While microsatellite locus 12 includes only five copies of a two nucleotide motif sequence, the microsatellite locus may include hundreds of copies of a 2 to 6 nucleotide motif, and it is preferable that the microsatellite locus have between 20 and 150 copies of a motif having between 2 and 6 nucleotides.

In general, DNA is composed of two antiparallel complementary strands. During DNA synthesis primed by primer 9 in this example, the upper strand is displaced and the lower strand is used as template with the complement (C pairs with G; A with T) of each base of the lower strand being sequentially added thereby synthesizing a new upper strand in the 5′ to 3′ direction. A dideoxy DNA sequencing reaction is prepared using DNA template 10. The sequencing reaction includes a primer, and the four deoxynucleotide triphosphates, dATP, dCTP, dGTP, and dTTP. In addition, the sequencing reaction includes dideoxy ATP (ddATP). The primer is labeled with a fluorescent tag, but could also be labeled by other means (e.g., radioactive tag, IR tag, etc.). The sequencing reaction is allowed to proceed (as indicated by arrow 16) to synthesize a two nucleotide ladder DNA size standard denoted 18.

During the sequencing reaction, DNA template 10 will be primed with primer 9 and a nascent DNA fragment will be synthesized, one nucleotide at a time, using the four deoxy nucleotides, dATP, dCTP, dGTP, and dTTP, and the dideoxy nucleotide, ddATP present in the sequencing reaction. Appropriate nucleotides will be added, one base at a time, to the last nucleotide (3′end) of a nascent DNA sequence and proceeds in the direction indicated by arrow 8.

When the appropriate nucleotide to be inserted is A (adenine), either dATP or ddATP will be added. More correctly, either dATP or ddATP will enter the synthesis reaction resulting in either dAMP (deoxyadenine monophosphate) or ddAMP being introduced into the elongating DNA chain. If dAMP is inserted, the chain can be further elongated by adding another nucleotide (e.g., cytosine). However, if a ddAMP is inserted, no additional nucleotides may be added. Therefore, elongation of the nascent DNA fragment terminates upon addition of ddATP.

The sequencing reaction proceeds on multiple copies of the DNA template, microsatellite locus 12. Randomly, a dideoxy ATP will be added to nascent DNA fragments at one of the A positions, thereby terminating the respective nascent fragment. Consequently, the nascent DNA fragments will have different lengths depending on the A position at which the dideoxy ATP was added.

At the conclusion of the DNA sequencing reaction, DNA size standard 18 is produced. DNA size standard 18 comprises five DNA fragments 20, 22, 24, 26 and 28 formed of the primer, 5′ flanking sequences and two to five copies of motif sequence 14, respectively. As shown, DNA fragments 20, 22, 24, 26 and 28 are 18, 20, 22, 24, and 26 nucleotides in length, respectively.

When a dideoxy ATP is randomly inserted at the first A of microsatellite locus 12, DNA fragment 20 is produced. Further, when a dideoxy ATP is randomly inserted at the second A, DNA fragment 22 is produced; similarly, at the third A, DNA fragment 24 is produced; at the fourth A, DNA fragment 26 is produced; and at the fifth A, DNA fragment 28 is produced.

In accordance with an important aspect of the invention, the microsatellite locus 12 with nucleotide motif sequence 14 is selected such that DNA fragments 20, 22, 24, 26, 28 do not form a respective secondary structure. The presence or lack of secondary structure can be determined empirically by analyzing the DNA sequence data using any of a number of techniques and computer software programs. For example, the Shareware program FOLD is one such program which analyzes a nucleotide sequence for secondary structure. Although FOLD was developed to calculate secondary structure of RNA molecules, it is used here as a method to approximate the relative amount of secondary structure in DNA. The FOLD program assigns energy values to the most stable intra-molecular species that can potentially form based upon the primary nucleotide sequence. Values, calculated at 50 degrees C., of −15 and smaller would indicate the presence of a secondary structure whereas values of −8.5 and larger would indicate the lack of a secondary structure. A further discussion on the FOLD program is provided by M. Zuker, “On Finding All Suboptimal Foldings of an RNA Molecule,” Science, 244, 48-52, (1989); J. A. Jaeger, D. H. Turner and M. Zuker, “Improved Predictions of Secondary Structures for RNA. Proc. Natl. Acad. Sci.,” USA, Biochemistry, 86, 7706-7710, (1989); and J. A. Jaeger, D. H. Turner and M. Zuker, “Predicting Optimal and Suboptimal Secondary Structure for RNA in “Molecular Evolution: Computer Analysis of Protein and Nucleic Acid Sequences,” R. F. Doolittle ed. Methods in Enzymology, 183, 281-306 (1989), all herein incorporated by reference.

The presence of secondary structure affects how a DNA fragment will migrate through an electrophoresis gel. By selecting a DNA template which lacks secondary structure, an accurate and consistent size standard can be provided which can be used to compare sizes and fragments across gels.

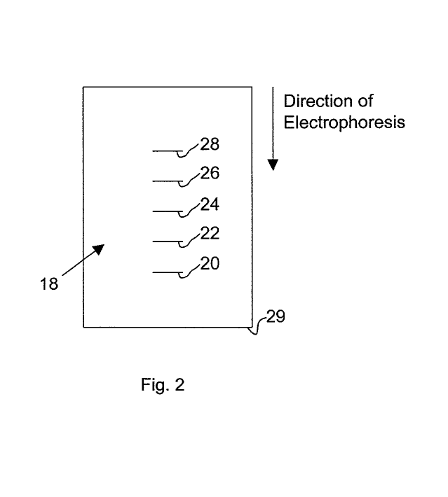

Referring now to FIG. 2, DNA size standard 18 is run through electrophoresis gel 29. The DNA fragments migrate according to their respective sizes. The DNA fragments 20, 22, 24, 26 and 28 migrate at uniform but different rates through electrophoresis gel 29. The rate of migration is inversely proportional to the fragment length since the fragments do not contain secondary structure. The distance DNA fragments migrate is inversely proportional to the length of the respective DNA fragments 20, 22, 24, 26, and 28 with shorter fragments migrating farther than longer fragments.

The present method may be modified in order to provide a DNA size standard with an alternate size difference between DNA fragments. For example, a three nucleotide motif sequence may be used to provide a three nucleotide DNA ladder. As such, the nucleotide DNA would have a three nucleotide difference between DNA fragments.

Further, the present method may be modified by choosing a microsatellite locus having more copies of the motif sequence. The resulting ladder will produce larger DNA fragments, which differ by the number of copies of the motif sequence size, up to the length of the microsatellite locus selected.

### EXAMPLE

In one example, chum salmon microsatellite clone C97 is used as a DNA template. C97 contains a microsatellite locus region having 60 copies of two nucleotide motif sequence CA.

A DNA size standard was produced using SequiTherm EXCEL II DNA sequencing kit (Epicentre, Madison, Wis.), using clone C97 DNA as the template, and a ddA (dideoxy ATP) terminator. The DNA template was primed using KS primer (Stratagene, San Diego, Calif.) that was labeled with a florescent tag (HEX).

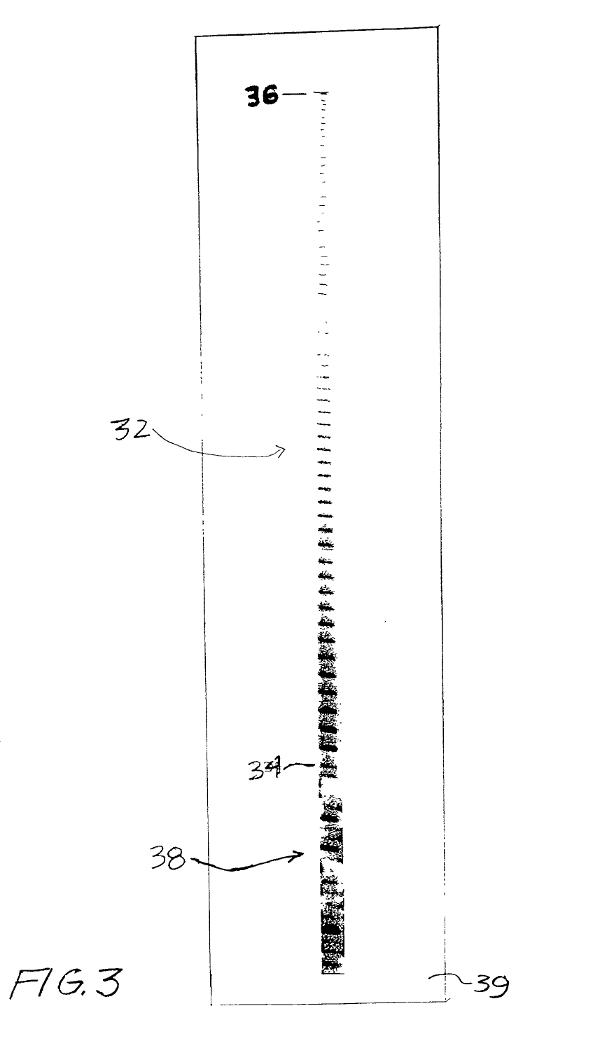

Referring now to FIG. 3, a two nucleotide ladder 32, produced by the sequencing reaction on C97, is shown which was run on gel 39. Two nucleotide ladder 32 provides a functional DNA size standard between a 71 nucleotide DNA fragment 34 and a 191 nucleotide DNA fragment 36, having fragments differing in length by two nucleotides.

DNA fragments having sizes larger than 191 nucleotides did not result in adequate visualization on gel 39. Although the spacing of DNA fragments smaller than 71 nucleotides is not 2 nucleotides, they can be used for sizing smaller fragments. However, other ladders that span smaller and larger size ranges can be synthesized to size smaller or larger fragments. Consequently, ladder 32 may be used to size DNA samples between 71 and 191 nucleotides long.

Although the invention has been described above in relation to preferred embodiments thereof, it will be understood by those skilled in the art that variations and modifications can be effected in these preferred embodiments without departing from the scope and spirit of the invention.

1. A method of producing DNA size standards, said method comprising the steps of:
providing a DNA template comprising multiple copies of a nucleotide motif sequence; and
preparing a dideoxy sequencing reaction using the DNA template and one dideoxy nucleotide terminator.

2. The method of claim 1, wherein the dideoxy nucleotide is selected from the group consisting of dideoxy ATP, dideoxy GTP, dideoxy CTP, dideoxy TTP and any analog thereof that can terminate the sequencing reaction.

3. The method of claim 1, wherein DNA fragments produced by the dideoxy sequencing reaction lack secondary structure.

4. The method of claim 1, wherein the motif sequence is one of a group consisting of two to six nucleotides.

5. The method of claim 1, wherein the DNA template comprises n copies of the motif sequence, wherein n is an integer from 10 to 200.

6. The method of claim 4, wherein the DNA template comprises n copies of the motif sequence, wherein n is an integer from 10 to 200.

7. The method of claim 1, wherein the DNA template comprises a microsatellite locus.

8. The method of producing a DNA size standard of claim 1, wherein the motif sequence comprises a sequence with at least one unique nucleotide base.

9. The method of producing a DNA size standard of claim 1, wherein the dideoxy sequencing reaction produces DNA fragments comprising a respective number of copies of the motif sequence

10. A DNA size standard, comprising:
a mixture of DNA fragments, each said DNA fragment being formed by a primer, 5′ sequence and a respective number of copies of a motif sequence.

11. The DNA size standard of claim 10, wherein each one of said DNA fragments differ in length from one of a next shorter length and from one of a next longer length by one said motif sequence.

12. The DNA size standard of claim 10, wherein said motif sequence comprises a sequence of a group consisting of two to six nucleotides.

13. The DNA size standard of claim 10, wherein said mixture comprises DNA fragments having m to n copies of said motif sequence, where m and n are integers.

14. The DNA size standard of claim 13, wherein n is a number from 10 to 200.

15. The DNA size standard of claim 14, wherein m is a number from 1 to n.

16. The DNA size standard of claim 10, wherein said motif sequence comprises a sequence of a group consisting of two to six nucleotides.

17. The DNA size standard of claim 10, wherein said motif sequence is CA.

18. The DNA size standard of claim 10, wherein said DNA fragments lack secondary structure.

19. The DNA size standard of claim 10, wherein said motif sequence comprises a sequence with at least one unique nucleotide base.
